(19) United States
(12) Plant Patent Application Publication
(10) Pub. No.: US 2023/0422650 P1
(43) Pub. Date: Dec. 28, 2023

(54) Caladium Plant Named 'Lava Glow'

(50) ;

(71) Applicant: Florida Foundation Seed Producers, Inc., Marianna, FL (US)

(72) Inventor: Zhanao Deng, Riverview, FL (US)

(21) Appl. No.: 18/212,943

(22) Filed: Jun. 22, 2023

Related U.S. Application Data
(60) Provisional application No. 63/354,405, filed on Jun. 22, 2022.

(51) Int. Cl. A01H 6/10

(52) U.S. Cl. PLT/373

(57) ABSTRACT

A new and distinct cultivar of Caladium plant named ‘Lava Glow’, characterized by its upright, full plant habit, numerous ovate to cordate shaped leaves that are peltate and ovate to cordate, palmate-pinnate leaf venation pattern, large red to reddish pink semi-translucent leaf centers, multiple pronounced red primary veins, numerous irregular red spots and blotches of varying sizes between the center and the leaf margin, multicolored spathes, tolerance to sunburns and leaf blight, and demonstrated plant performance in containers and gardens.

### CROSS-REFERENCE TO RELATED APPLICATIONS

This Application is related to and claims the benefit of U.S. Provisional Patent Application Ser. No. 63/354,405, filed on Jun. 22, 2022, entitled CALADIUM CULTIVARS, the entirety of which is incorporated herein by reference.

Genus and species: Caladium x hortulanum.

Cultivar denomination: ‘Lava Glow’.

### BACKGROUND OF THE NEW CULTIVAR

The present invention relates to a new and distinct cultivar of Caladium, botanically known as Caladium x hortulanum, and hereinafter referred to by the name ‘Lava Glow’. Caladium plants are tuberous-rooted plants and typically have attractive heart-shaped or arrow-shaped leaves on long stalks rising directly from the tubers. Foliage may grow in a variety of colors and variegation patterns, and many species have been developed for aesthetic coloration, good container and landscape growth, drought tolerance, pest resistance, and other desirable characteristics.

Plant Breeder's Rights for this cultivar have not been applied for. The new Caladium cultivar ‘Lava Glow’ has not been made publicly available more than one year prior to the filing of this application.

### SUMMARY OF THE INVENTION

The new Caladium cultivar ‘Lava Glow’ has not been observed under all possible environmental conditions. The phenotype of the new cultivar may vary with variations in environment and cultural practices such as temperature, light intensity, fertilization, irrigation, and application of plant growth regulators without any change in genotype.

The objective of the breeding program that produced the present cultivar was to develop cultivars that have novel foliar characteristics, produce multiple brightly colored leaves, have good tuber yield potential, and perform well in containers and the landscape. The present Caladium cultivar originated from a cross-pollination made in Balm, Florida, in the summer of 2013 between the Caladium x hortulanum cultivar ‘Fire Chief’ (not patented), as the female or seed parent, and the Caladium x hortulanum cultivar ‘UF 4412’ (U.S. Plant Pat. No. 25,612, commercial name “Fairytale Princess”), as the male or pollen parent. The new Caladium cultivar was discovered and selected by the inventor as a single plant within the progeny of the stated cross-pollination in Balm, Florida, in the fall of 2015. ‘UF 4412’ was a progeny of a cross made between the Caladium x hortulanum variety ‘Florida Sweetheart’ (U.S. Plant Pat. No. PP8,526; patent expired) and the Caladium x hortulanum variety ‘Red Flash’ (not patented).

Asexual reproduction of the new cultivar ‘Lava Glow’ by tuber divisions has been performed annually since 2016. The unique features of this new Caladium cultivar are stable and reproduced true to type in successive generations of asexual reproduction.

The new cultivar ‘Lava Glow’ has not been observed under all possible environmental conditions. The phenotype may vary somewhat with variations in environment such as temperature and light intensity, without, however, any variance in genotype.

The following traits have been repeatedly observed and are determined to be the unique characteristics of ‘Lava Glow’. These characteristics in combination distinguish ‘Lava Glow’ as a new and distinct cultivar of Caladium:
1. Upright growth habit with intermediate plant heights;
2. Vigorous growth with a dense foliage canopy having numerous leaves;
3. Ovate to cordate shaped leaves having a mostly red to reddish pink coloration with splotches of green mixed with red and reddish pink nearer to the margins;
4. Rich red venation coloration and dark green margins;
5. Multicolored blooms or spathes; and
6. Tolerance to sunburns and leaf blight disease

Plant of the new Caladium cultivar ‘Lava Glow’ differ from plants of the female parent, ‘Fire Chief’, in the following characteristics:
1. Plants of ‘Lava Glow’ are fuller and more attractive than plants of ‘Fire Chief’;
2. Leaves of ‘Lava Glow’ have a broader translucent reddish pink center;
3. The mid leaf vein of ‘Lava Glow’ is red to reddish pink, whereas ‘Fire Chief’ has a green mid leaf vein; and
4. The center of the leaves of ‘Lava Glow’ is mostly translucent.

Plants of the new Caladium cultivar ‘Lava Glow’ differ from plants of the male parent, ‘UF 4412’, in the following characteristics:
1. Plants of ‘Lava Glow’ are taller and larger than those of ‘UF 4412’;
2. Leaves of ‘Lava Glow’ are peltate, each leaf having two large lobes;
3. Leaves of ‘Lava Glow’ are longer and wider than those ‘UF 4412’:
4. Leaves of ‘Lava Glow’ have numerous green spots toward the margins; and
5. The center of the leaves of ‘Lava Glow’ is mostly translucent.

Plants of the new Caladium ‘Lava Glow’ can also be compared to plants of the commercial cultivar ‘Brandywine’ (not patented). Plants of ‘Lava Glow’ differ primarily from plants of ‘Brandywine’ in leaf color, mid vein color, and coloration of the bloom. Plants of ‘Brandywine’ have wider leaves than those of ‘Lava Glow’. Leaves of ‘Brandywine’ are mainly solid red in color with few green splotches. The mid veins on leaves of ‘Brandywine’ are not as pronounced as mid veins of ‘Lava Glow’. The spathes of ‘Brandywine’ are mostly white and creamy to light green on top, while ‘Lava Glow’ has spathes that are greenish white infused with splotches of red.

### DESCRIPTION OF THE FIGURES

The accompanying photographs (as shown in FIGS. 1-9) illustrate the overall appearance of the new Caladium cultivar ‘Lava Glow’. These photographs show the colors as true as can be reasonably obtained in colored reproductions of this type. Colors in the photographs may differ slightly from the color values cited in the detailed botanical description, which accurately describe the colors of the new Caladium cultivar.

FIG. 1 shows a side perspective view of a typical plant of ‘Lava Glow’ grown from four No.1-sized (3.6 to 6.4 cm diameter) tubers forced in a 20-cm container and grown for five to eight weeks in a greenhouse;

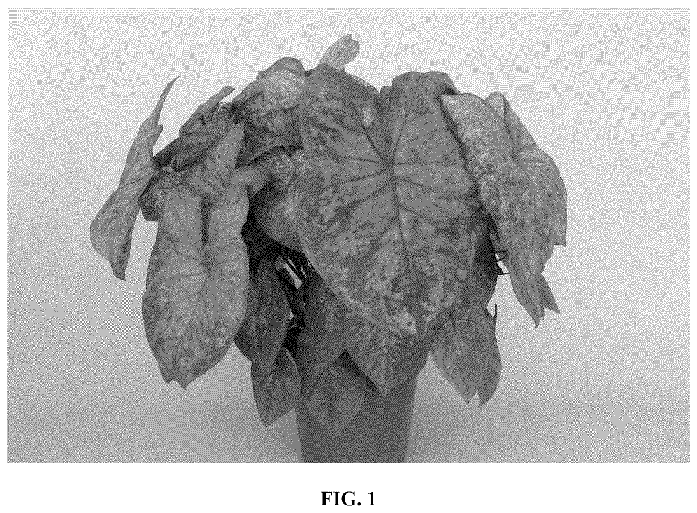

FIG. 2 shows a side perspective view of typical plants of ‘Lava Glow’ (left) and its closest counterpart commercial cultivar ‘Brandywine’ (right) forced from four No.1-sized (3.6 to 6.4 cm diameter) in 20-cm diameter containers and grown in a greenhouse for five to eight weeks;

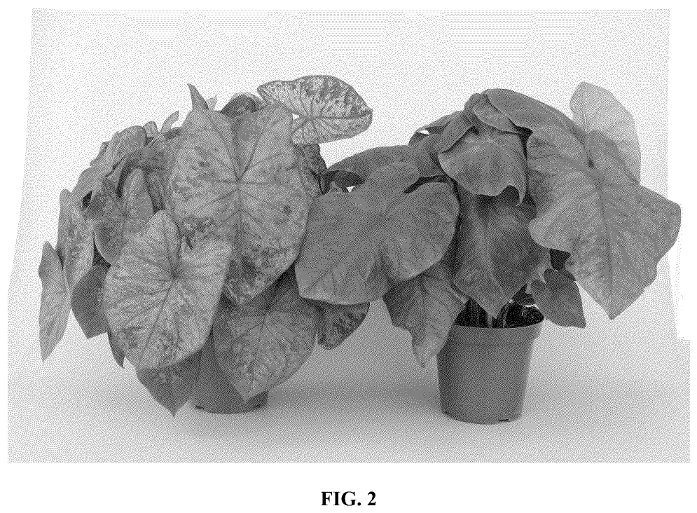

FIG. 3 shows a side perspective view of a typical mature leaf of a ‘Lava Glow’ plant forced from tubers in a 20-cm diameter container and grown in the greenhouse for five to eight weeks;

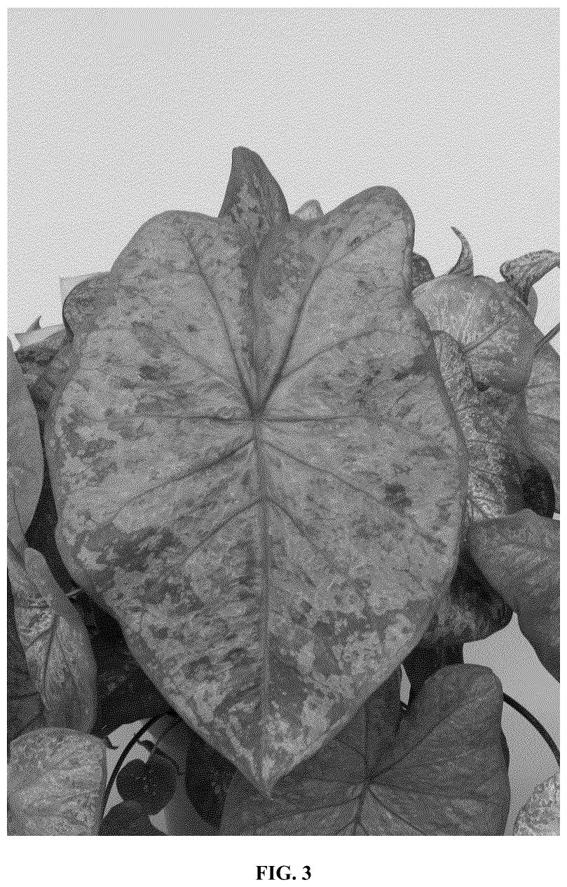

FIG. 4 shows a side view of the underside of a typical mature leaf of a ‘Lava Glow’ plant forced from tubers in a 20-cm diameter container and grown in the greenhouse for five to eight weeks;

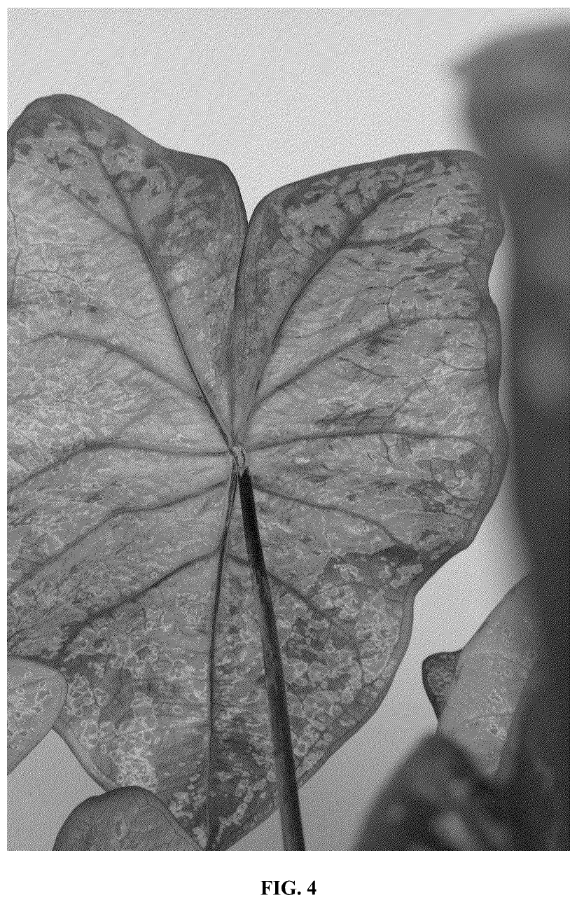

FIG. 5 shows a top view of typical mature leaves of ‘Lava Glow’ (two leaves on the left) and its closest commercial counterpart ‘Brandywine’ (two leaves on the right);

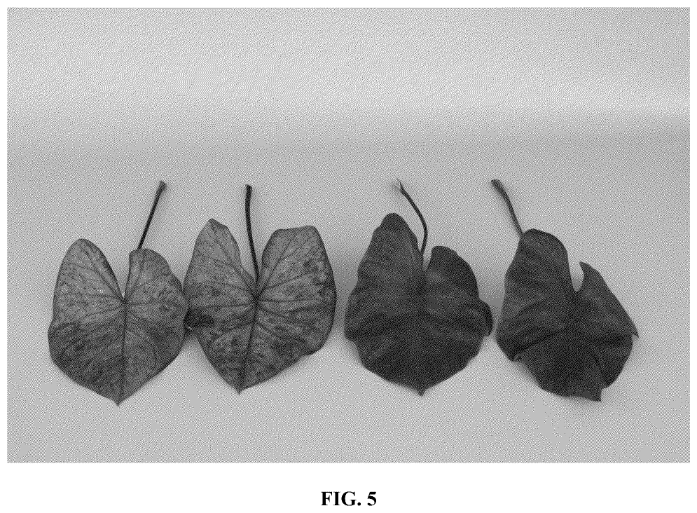

FIG. 6 shows a top view of the underside of typical mature leaves of ‘Lava Glow’ (two leaves on the top) and its closest commercial counterpart ‘Brandywine’ (two leaves on the bottom);

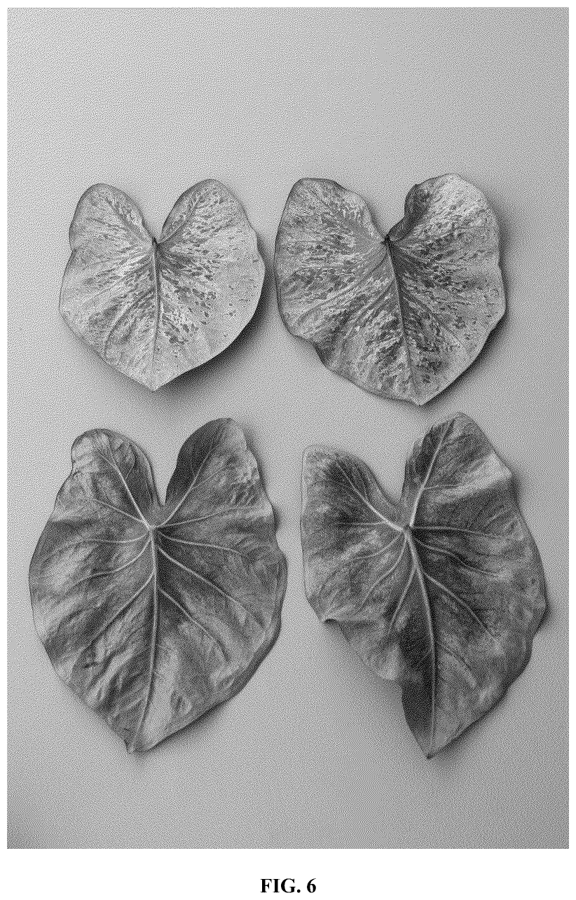

FIG. 7 shows a side view of an open spathe of ‘Lava Glow’;

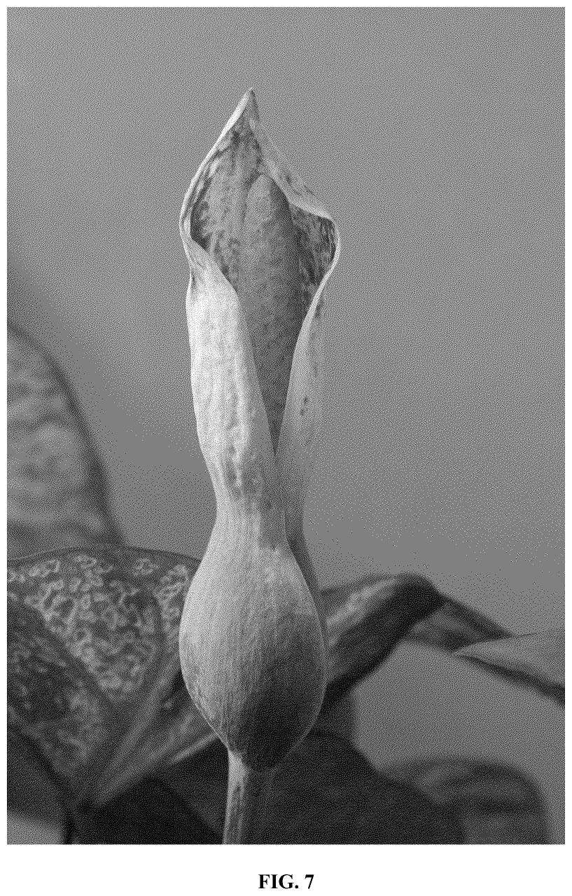

FIG. 8 shows a side view of an open spathe and exposed spadix of ‘Lava Glow’; and

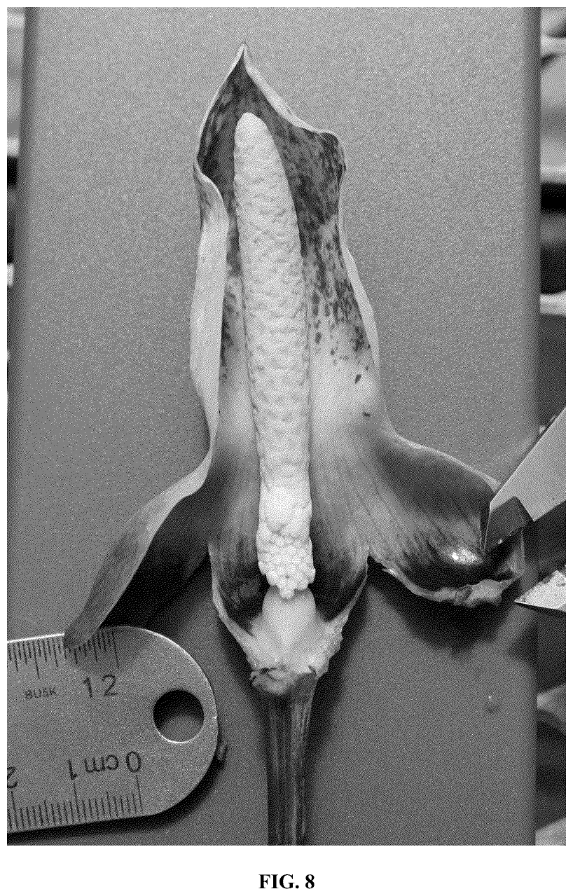

FIG. 9 shows a top view of plants of ‘Lava Glow’ that were grown in full sun in an open field in central Florida for 4-5 months to demonstrate tolerance to sunburns and leaf blight disease.

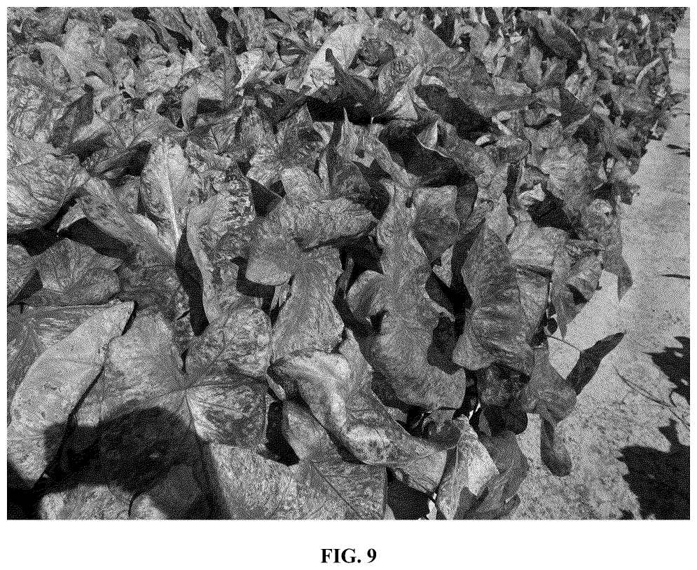

### DETAILED BOTANICAL DESCRIPTION OF THE CULTIVAR

In the following description, color references are made to The Royal Horticultural Society (R.H. S.) Colour Chart, 1986 Edition, except where general terms of ordinary dictionary significance are used.

### Description of Growing Conditions

The aforementioned photographs and the following observations and measurements describe plants grown in 20-cm containers in Balm, Florida, during the summer in a polypropylene-covered shade house. All plants were grown under conditions and practices that approximate those generally used in commercial Caladium production.

During the production of the plants, day temperatures ranged from about 26° C. to 35° C., night temperatures ranged from about 18° C. to 26° C. and light levels ranged from 2400 to 3840 foot-candles in the greenhouse. Plants grown in the greenhouse were six to seven weeks old from planting tubers when the photographs and the detailed description were taken.
Botanical description:
   Botanical classification.—Family: Araceae. Botanical name: Caladium x hortulanum. Common name: Caladium or Caladium. Cultivar: ‘Lava Glow’.
   Parentage.—Female or seed parent: Caladium x hortulanum cultivar ‘Fire Chief’ (not patented). Male or pollen parent: Caladium x hortulanum cultivar ‘UF 4412’ (U.S. Plant Pat. No. 25,612).
   Propagation.—Type: By tubers and by tuber divisions. Time to initiate roots, summer: About seven to ten days at 32° C. Time to initiate roots, winter: About two to three weeks at 24° C.
   Tuber description.—Number of buds per No. 1-sized (approximately 3.7 to 6.4 cm in diameter) tuber: Greenhouse grown plants: About six to nine actively growing buds/shoots. Outdoor nursery-grown plants: About four to six actively growing buds/shoots. Height: 4.0 cm. Diameter: 3.7 to 6.4 cm. Texture: Thick, starchy, and hairy with fine dried roots. Color: Epidermis: Between RHS 200C and 200D (brown). Interior: A mixture of RHS 10B, 10C and 10D (yellow), mostly 10B.
   Root description.—Dense, thick, and white fleshy roots.
   Plant description.—Type: Herbaceous perennial. Growth habit: Vigorous, upright, dense growth habit with an abundance of leaves. suitable for growing in 10 to 30-cm sized containers. Leaf petioles: Arising from tubers, mostly upright, curving outwardly with development. Plant height, from soil level to top of leaf plane: About 43.3 cm. Plant height, from soil level to top of inflorescences: About 37.3 cm. Plant diameter or spread: Average 67.0 cm.
   Foliage description.—Length: About 27.3 cm. Width (flattened): About 18.3 cm. Shape: Ovate to cordate, peltate. Apex: Acuminate. Base: Ovate to cordate. Margin: Entire. Texture, upper surface: Smooth, glabrous. Texture, lower surface: Smooth, glabrous; glaucous. Venation pattern: Palmate to pinnate. Color, developing leaves: Upper surface: Center: RHS 52B (pink) with a few splotches of RHS 53B (red). Borders and margins: RHS 137A (green), with a thin line of RHS 53A (red) on the extreme edge of the margins. Basal notch: RHS 53A (red). Venation (both mid rib and primary veins): RHS 53A (red) with RHS 53B (red) slightly outward from the primary veins. Lower surface: Center: A mixture of RHS 58A and 58B (red-purple). Border and margins: RHS 138B (green). Venation, midrib: RHS 58A (red-purple). Venation, primary veins: RHS 58B (red-purple). Color, fully expanded leaves: Upper surface: Center: A mixture of RHS 53A, 53B, and 53C (red) with some RHS 40B (reddish orange). Outwards from the center and towards the margins: Numerous irregular sized spots and blotches of RHS 40C (red). Borders and margins: RHS 137A (green) with a thin line of RHS 53A (red) on extreme edge of the margins. Basal notch: RHS 53A (red). Venation (both mid rib and primary veins): RHS 53A (red) with some RHS 53B slightly outward from the primary veins. Lower surface: Center: RHS 59A (red-purple). Border and margins: RHS 139C(green), with much larger green margins on the lower surface of the leaf than the upper surface. Outwards from the center towards the margins: Numerous small botches and spots of RHS 61C and 61D (red-purple). Venation (both mid rib and primary veins): RHS 59B (red-purple). Petiole: Aspect: Mostly erect, curving outwardly with development. Length: About 31.5 cm. Diameter, distal: About 4.4 mm. Diameter, proximal: About 7.6 mm. Strength: Strong, flexible. Color: A mixture of RHS 200A, 200B, and 200C (brown), variably streaked with RHS 200D (light brown), RHS 79D (light Purple), or RHS 59A (red-purple), younger leaves have lighter petioles of RHS 59C and 59D (red-purple). Wing length: About 6.7 cm. Wing diameter: About 6.7 cm. Wing color: RHS 200B (brown), streaked with RHS 200C (brown) and RHS 177C (greyed orange), wings on young leaves have light streaks of RHS 177C and 177D (reddish orange).
   Inflorescence description.—Arrangement: Upright hooded spathes surrounding a columnar spadix borne on a tall upright scape. Spadix with sessile, simple female and male flowers separated into two zones: female flowers arranged on the lower one-third of the spadix, male flowers arranged on the upper two-thirds of the spadix. Sterile flowers develop between female and male flower zones; near this area, the spathe constricts surrounding the female flowers. Fragrance: None detected. Natural flowering season/longevity: Spring or early summer in central Florida; flowers develop about seven weeks after growth commences; inflorescences last about three days before fading; persistent. Spathe: Length: About 11.7 cm. Width, distal: About 2.8 cm. Width, proximal: About 2.4 cm. Shape: Ovate to obovate. Apex: Acute to acuminate. Base: Tapering. Margin: Entire; proximal, notched. Texture, upper and lower surfaces: Smooth, glabrous. Color: Front surface: Upper two-thirds: RHS 159B and 159C (orange-white), with streaks and splotches of RHS 45A and 45B (red), RHS 60A (red-purple), or RHS 186C (greyed purple). Lower one-third: RHS 143A, 143B, and 143C (green), with numerous splotches and streaks of RHS 45A and 45D (red), RHS 58A and 58C (red- purple), RHS 60B and 60C (red-purple), or RHS 186C (greyed purple). Rear surface: Upper two-thirds: RHS 145A and 145C (yellow-green) to RHS 159A, 159B 159C, and 159D (orange-white), with numerous splotches and streaks of RHS 45A and 46A (red) and RHS 186A, 186B, and 186C (greyed purple). Lower one third: Varies from RHS 143B (green), RHS 145A (yellow-green) to RHS 58A (red-purple), and heavily streaked and dotted with RHS 45A, 45B, 45C, and 45D (red), RHS 60A and 60B (red-purple), RHS 186C (greyed purple), or RHS 159B (orange-white). Spadix: Length, entire spadix: About 7.6 cm. Shape: Spindle-shaped to columnar. Apex: Obtuse. Base: Obtuse. Aspect: Upright. Male flower zone: Length: About 5.6 cm. Diameter: About 9.7 mm. Color, mature: RHS 161B and 161C (greyed-yellow) or RHS 145B (yellow-green). Male flowers: Quantity per spadix: About 190. Shape: Irregularly diamond shaped. Height: About 4.0 mm. Diameter: About 2.8 mm. Anther color: From RHS 160A (greyed yellow) and RHS 158A (yellow-white) to RHS 145A and 145B (yellow-green). Amount of pollen: Good amount of pollen on most blooms. Sterile flower zone: Length: About 1.25 cm. Diameter: About 7.6 mm. Color, mature: RHS 159A and 159B (orange-white). Female flower zone: Length: About 0.85 cm. Diameter: About 7.4 mm. Color: RHS 160B (greyed yellow) to RHS 159A (orange-white). Female flowers: Quantity per spadix: About 37. Shape: Slightly hexagon or round. Height: About 2.2 mm. Diameter: About 2.0 mm. Stigma color: RHS 160B and 160C (greyed yellow) to RHS 159B (orange-white). Ovary color: RHS 160A and 160B (greyed yellow) to RHS 159A (orange-white). Scape: Length: About 26.4 cm. Diameter: About 5.5 mm. Strength: Sturdy, strong and flexible. Aspect: Erect. Texture: Smooth, glabrous; glaucous. Color: RHS 183A, 183B, 183C, and 185A (greyed purple) to RHS 200A (brown), densely streaked with RHS 46A, 46B (red) and RHS 183A, 183B, 183C, and 183D (greyed purple). Just below spathe: RHS 143C (green), RHS 144C, 145A (yellow-green), and RHS 160C (greyed yellow) to RHS 200D (brown), with numerous streaks of RHS 183A 183B, 183C, and 183D (greyed purple), or RHS 58A (red-purple). Seeds and fruit: Seed and fruit development has not been observed.
Disease resistance: Plants of the new Caladium cultivar ‘Lava Glow’ have been observed to be less susceptible to Fusarium tuber rot caused by Fusarium solani than the commercial Caladium cultivars ‘Carolyn Whorton’ (not patented) and ‘Postman Joyner’ (not patented). Plants of the new Caladium cultivar ‘Lava Glow’ have been observed to be more resistant to leaf blight disease and have healthier leaves than the commercial Caladium cultivars ‘Freida Hemple’ (not patented) and ‘Brandywine’ (not patented).
Sunburn resistance: Plants of the new Caladium cultivar ‘Lava Glow’ have been observed to show better tolerance to sunburns than the widely grown red fancy-leaved ‘Freida Hemple’ and the commercial Caladium cultivar ‘Brandywine’. In replicated field trials in Florida, the sunburn tolerance score of ‘Lava Glow’ ranged from 3.3 to 4.8 on a scale of 1 to 5, with 1=highly sensitive and 5=highly tolerant to sunburns. In the same trials, the widely grown commercial cultivar ‘Freida Hemple’ scored 1.2 to 4.0 on the same scale of 1 to 5.

We claim:

1. A new and distinct cultivar of Caladium plant named ‘Lava Glow’ as illustrated and described herein.
